(19) United States
(12) Patent Application Publication
(10) Pub. No.: US 2021/0023308 A1
(43) Pub. Date: Jan. 28, 2021

(54) TRANSMISSION PROTOCOL FOR MEDICAL DEVICE WITH LOGGING FEATURE

(71) Applicant: Novo Nordisk A/S, Bagsvaerd (DK)

(72) Inventor: Per Einar Pontus Holm, Malmoe (SE)

(21) Appl. No.: 16/977,409

(22) PCT Filed: Mar. 7, 2019
(86) PCT No.: PCT/EP2019/055762

§ 371 Date: Sep. 1, 2020

(30) Foreign Application Priority Data
Mar. 9, 2018 (EP) 18160952.0

(51) Int. Cl. A61M 5/315; G16H 20/17; G16H 40/67

(52) U.S. Cl. CPC A61M 5/31535; G16H 40/67; G16H 20/17

(57) ABSTRACT

The invention provides a method for wirelessly communicating a dynamic data log from a data generating device using a transmit-only protocol is provided, the dynamic data log comprising at least one latest data entry, and a plurality of previous data entries. The method comprises the steps of continuously or intermittently transmitting the dynamic data log as a plurality of data packets, wherein the data packets comprise a prioritised packet populated by the at least one latest data entry, and a plurality of regular segment packets, each being populated with a subset of the plurality of previous data entries. The prioritised packet is transmitted more frequently than at least one of the regular segment packets.

The present invention generally relates to methods and devices for wirelessly communicating a dynamic data log from a data generating device, e.g. to medical devices for which the generation, collecting and storing of data are relevant. In specific embodiments the invention relates to devices and systems for capturing and transmitting drug delivery dose data in a reliable and user-friendly way.

### BACKGROUND OF THE INVENTION

In the disclosure of the present invention reference is mostly made to drug delivery devices comprising a threaded piston rod driven by a rotating drive member, such devices being used e.g. in the treatment of diabetes by delivery of insulin, however, this is only an exemplary use of the present invention as it may be implemented in any given technical field in which the transfer of a dynamic data log is relevant, e.g. for medical devices in general in which drugs are administered or in which physiological data is measured and logged.

Drug Injection devices have greatly improved the lives of patients who must self-administer drugs and biological agents. Drug Injection devices may take many forms, including simple disposable devices that are little more than an ampoule with an injection means or they may be durable devices adapted to be used with prefilled cartridges. Regardless of their form and type, they have proven to be great aids in assisting patients to self-administer injectable drugs and biological agents. They also greatly assist care givers in administering injectable medicines to those incapable of performing self-injections.

Performing the necessary insulin injection at the right time and in the right size is essential for managing diabetes, i.e. compliance with the specified insulin regimen is important. To make it possible for medical personnel to determine the effectiveness of a prescribed dosage pattern, diabetes patients are encouraged to keep a log of the size and time of each injection. However, such logs are normally kept in handwritten notebooks, and the logged information may not be easily uploaded to a computer for data processing. Furthermore, as only events, which are noted by the patient, are logged, the note book system requires that the patient remembers to log each injection, if the logged information is to have any value in the treatment of the patient's disease. A missing or erroneous record in the log results in a misleading picture of the injection history and thus a misleading basis for the medical personnel's decision making with respect to future medication. Accordingly, it may be desirable to automate the logging of injection information from medication delivery systems.

Correspondingly, some proposed drug delivery devices integrate this monitoring/acquisition mechanism into the device itself, e.g. as disclosed in US 2009/0318865, WO 2010/052275 and WO 2016/110592, these devices being of the durable type, whereas WO 2015/071354 discloses a disposable drug delivery device provided with dose logging circuitry.

However, most devices of today are without it. Addressing this problem, many solutions have been proposed which would help a user to generate, collect and distribute data indicative of the use of a given medical device. For example, WO 2013/120776 describes an electronic supplementary device (or add-on device) adapted to be releasably attached to a drug delivery device of the pen type. The device includes a camera and is configured to perform optical character recognition (OCR) on captured images from a rotating scale drum visible through a dosage window on the drug delivery device, thereby to determine a dose of medicament that has been dialled into the drug delivery device. A further external device for a pen device is shown in WO 2014/161952, the external device being designed to determine dose sizes based on detection of movement of a magnetic member incorporated in the pen device.

Although the above-described logging devices in general are provided with a display allowing logged dose data to be displayed, it may be desirable to transfer dose data to an external device, e.g. a smartphone as carried by many drug delivery device users, this allowing the dose data to be displayed on a much larger display and to be further processed and used for e.g. analysis and recommendations. Such an arrangement would also allow the display on the logging device to be dispensed with. WO 2016/108888 discloses a temperature logging patch adapted to transmit the log in a number of data packets, the most recent temperature value being included in each packet.

Having regard to the above, it is an object of the present invention to provide devices and methods allowing efficient and cost-effective wireless transfer of a dynamic data log from a data generating device, e.g. from a drug delivery device or from a physiological sensor device with logging capabilities to an external device such as a smartphone.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention a method for wirelessly communicating a dynamic data log from a data generating device using a transmit-only protocol is provided, the dynamic data log comprising a most-recent data entry, and a plurality of previous data entries. The method comprises the steps of continuously or intermittently transmitting the dynamic data log as a plurality of data packets, wherein the data packets comprise a prioritised packet populated by the most-recent data entry, and a plurality of regular segment packets, each being populated with a subset of the plurality of previous data entries, wherein the prioritised packet is transmitted more frequently than at least one of the regular segment packets.

By this arrangement secure and cost-effective wireless transfer of a dynamic data log from a data generating device can be provided in a user-friendly way, e.g. from a medical device with dose logging or parameter sampling capabilities to an external device such as a smartphone. Especially, the method assures that the user in a time effective way can be provided with the most recent information, e.g. data in respect of the last dose or the last few doses of drug expelled from a drug delivery or the latest data value(s) sampled by a sensor device, while at the same time also the remaining log or data entries can be transferred efficiently.

The dynamic data log may further comprise at least one recent data entry generated immediately before the most-recent data entry, wherein the prioritised packet is populated by the mostrecent data entry and the at least one recent data entry. Indeed, in the above general disclosure of the first aspect of the invention the recent data entries form part of the previous data sets.

Most of the time only the most recent dose(s) is/are of immediate interest, therefore the protocol priorities the response time for the most recent dose(s) at the cost of longer time to transfer the whole log by transmitting the most recent dose(s) more often than the older doses. This said, the method provides that timely transmission of the most recent data can be achieved while at the same time not sacrificing efficient transfer of the entire data log.

As appears, the above-described method defines that data entries are classified according to their “age”. For example, for a log of 20 data entries with entry 1 being the most recent, data entry 1 would be classified as the most-recent data entry, data entries 2-5 could be classified as recent data entries, with data entries 6-20 being classified as previous data entries. Correspondingly, for a log of e.g. only 4 data entries, data entry 1 would be classified as the mostrecent data entry, with the 3 data entries 2-4 being classified as recent or previous data entries. Thus, for small data logs some data classifications may not come in use and some types of data packages may be “empty” and then not created or transmitted.

In an exemplary embodiment the prioritised packet and the regular segment packets are transmitted according to a predetermined order. The regular segment packets may be transmitted according to a dynamic order, the dynamic order being randomized or determined based on the number of data entries in the dynamic data log.

In exemplary embodiments the regular segment packets are populated with a subset of the plurality of previous data entries according to a predetermined order. Alternatively, for each transmission the population of the regular segment packets with previous data entries may be randomized.

In exemplary embodiments the dynamic data log further comprises at least one last data entry generated immediately before the at least one recent data entry. The data packets further comprise one or more last segment packets populated by the at least one last data entry. The plurality of regular segment packets is each populated with a subset of the data entries not included in the prioritised packet or the last segment packet, and each last segment packet is transmitted more frequently than at least one of the regular segment packets.

Corresponding to the above-given example, for a log of e.g. 30 data entries, data entry 1 would be classified as the most-recent data entry, data entries 2-5 could be classified as recent data entries, with data entries 6-10 being classified as last data entries, and with data entries 11-30 being classified as previous data entries.

In general, the last segment packet and/or a regular segment packet may be transmitted only when being populated with at least one data entry, i.e. no “empty” packets are transmitted.

The prioritised packet may comprise a message authentication code for at least one of (i) the prioritized packet, and (ii) the whole data log. In the first case this would allow the most-recent data entry to be received also in case of non-successful transmission of the remaining log.

The last segment packet(s) may also comprise a message authentication code for at least one of (i) the last segment packet, and (ii) the whole data log. In the first case this would allow the last data entries to be received also in case of non-successful transmission of the remaining log.

The prioritised packet may be in the form of a header packet further comprising data indicating one or more of: identity of the data generating device, properties of the data generating device, and properties and/or type of the data entries.

In an exemplary embodiment transmission of data packets takes place in an active mode and an idle mode. In the active mode the entire data log is transmitted with the data packets being transmitted at a first rate. In the idle mode only the prioritised packet is transmitted, the prioritised packet being transmitted at a second rate, the second rate being lower than the first rate. The first transmission rate may be with intervals less than a second and the second transmission rate may be with intervals of more than a second.

The log will typically be in the form of a number of events comprising data representing a dose amount in combination with a time value. The stored data may be in the form or raw data only, e.g. rotational increments, this allowing the receiving unit, e.g. a smartphone or PC, to calculate the actual drug dose amounts based on supplied information in respect of the type of drug, type of cartridge, and type of device.

In a specific aspect of the invention a drug delivery device is provided, the drug delivery device comprising a drug reservoir or means for receiving a drug reservoir, drug expelling means comprising dose setting means allowing a user to set a dose amount of drug to be expelled, and electronic circuitry adapted to create a dynamic data log related to expelled dose amounts of drug. The electronic circuitry comprises sensor means adapted to capture a property value related to the dose amount of drug expelled from a reservoir by the expelling means during an expelling event, storage means adapted to store a plurality of property values to create the dynamic log, the dynamic log comprising, with a sufficient amount of property values having been created: at least one latest data entry, and a plurality of previous data entries, as well as transmission means for wireless transfer of the dynamic data log to an external device, the transmission means being configured to transfer the dynamic data log using a transmit-only protocol as described above.

In a further specific aspect of the invention a drug delivery device is provided, the drug delivery device comprising a drug reservoir or means for receiving a drug reservoir, drug expelling means comprising dose setting means allowing a user to set a dose amount of drug to be expelled, and electronic circuitry adapted to create a dynamic data log related to expelled dose amounts of drug. The electronic circuitry comprises sensor means adapted to capture a property value related to the dose amount of drug expelled from a reservoir by the expelling means during an expelling event, storage means adapted to store a plurality of property values to create the dynamic log, the dynamic log comprising, with a sufficient amount of property values having been created, at least one latest data entry, and a plurality of previous data entries, as well as transmission means for wireless transfer of the dynamic data log to an external device. The transmission means is configured to transfer the dynamic data log using a transmit-only protocol in which transmission of data packets takes place in an active mode and an idle mode as described above. The transmission means can be operated in accordance with the active mode and the idle mode, with the transmission means being operated in the active mode for a predetermined amount of time when a data log entry has been created and stored, after which the transmission means is operated in the idle mode.

Although a given device may be configured to transfer a dynamic data log using a transmitonly protocol as described above, the electronic circuitry of such a device may be configured to also provide two-way communication, e.g. when establishing pairing with a given external device.

In a yet further specific aspect of the invention a sensor device is provided, the sensor device comprising sensor means adapted to determine a physiological property value, and electronic circuitry adapted to create a dynamic data log related to determined physiological property value. The electronic circuitry comprises storage means adapted to store a plurality of physiological property values to create the dynamic log, the dynamic log comprising, with a sufficient amount of physiological property values having been created: at least one latest data entry, and a plurality of previous data entries, as well as transmission means for wireless transfer of the dynamic data log to an external device. The transmission means is configured to transfer the dynamic data log using a transmit-only protocol as described above.

The sensor device may be an external device adapted to be mounted e.g. on a skin surface and adapted to measure and log a physiological parameter such as blood glucose values or skin temperatures, or it may be in the form of a device adapted to be implanted, e.g. a pacemaker adapted to measure and log electrocardiographic values.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof. In the description of exemplary embodiments reference will be made to the use of insulin, however, the described module could also be used to create logs for other types of drug, e.g. growth hormone or drugs for haemophilia treatment.

### BRIEF DESCRIPTION OF THE DRAWINGS

In the following embodiments of the invention will be described with reference to the drawings, wherein

FIG. 1 shows a first example where a dose log with 21 records is being transmitted,

FIG. 2 shows a second example where a dose log with 21 records is being transmitted,

FIG. 3A shows a first drug delivery device,

FIG. 3B shows a flexible sheet with electronic circuitry,

FIG. 4 shows a second drug delivery device, and

FIG. 5 shows an add-on device mounted on a third drug delivery device.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

The present invention addresses the general issue of providing secure, easy and cost-effective wireless transfer of a dynamic data log from a data generating device to an external device. In a first exemplary embodiment timely, seamless, and cost effectively transfer from the data generating device to the external device is accomplished using a custom Bluetooth® Low Energy (BLE) radio chip. By removing the receiver part of the radio, the size and complexity of the radio chip can be significantly reduced and thus the cost. Such a radio chip may be incorporated in a drug delivery pen device with dose logging capabilities, this allowing for secure, easy and cost-effective wireless transfer of dose log data from the pen device to e.g. a mobile device such as a smartphone or a tablet computer.

With such a set-up, there is no handshake so the external device cannot query for e.g. the specific data it may lack due to previously non-received data. The data generating device must thus continuously transfer the whole log. After a data set has been generated, the user should receive the data within a short period of time, especially in case the data generating device is not provided with display means. However, to save energy the radio should transmit as seldom as possible.

Addressing these issues, the present inventors have realized that in most of the cases the user is primarily interested in the most recent information, e.g. data in respect of the last dose or the last few doses of drug expelled from a drug delivery. Correspondingly, these data can be prioritized by being transmitted more frequently to the cost of a slightly longer transfer time for the whole log. In this way shorter response times with less power consumption compared to round-robin transmission of e.g. a dose log can be achieved. Further, the transmit-only implementation reduces chip area by removing the need for receiver circuitry and simplifies development of software for a BLE stack, thereby potentially reducing cost significantly.

In a specific implementation of the invention, a drug delivery pen device with dose logging functionality broadcasts the dose log using the manufacturer specific data field in the BLE advertising packets. Because of the one-directional communication the device cannot receive acknowledgments so the whole dose log needs to be transmitted every time.

Most of the time only the last dose or the few last doses are of immediate interest, therefore the protocol priorities the response time for the latest doses at the cost of longer time to transfer the whole log by transmitting the latest dose more often than the oldest doses.

In a specific implementation the protocol makes use of two distinct packet formats, a header (or priority) packet and a segment packet. The header packet identifies the device as a “Type X” pen device and contains general information such as number of log entries, drug type as well as the last dose record. The segment packet contains a segment with e.g. up to 5 dose records of the dose log.

In a tested implementation the packets are interleaved according to the following scheme: Every 3rd packet sent is a header packet, every 4^{th}/5^{th} packet is the latest segment packet (e.g. comprising the latest 5 dose records), and remaining packets are segments from the log in randomized order. The packet sequence is thus: HLSHSLHSS (H—header packet, L latest segment, S—other segment). Packets may be sent only as needed so in the beginning of a pen life when only one dose is taken, only header packets are sent, then for the next 5 injections only header packets interleaved with last segment packets are sent. The header packet may also be termed a prioritised packet.

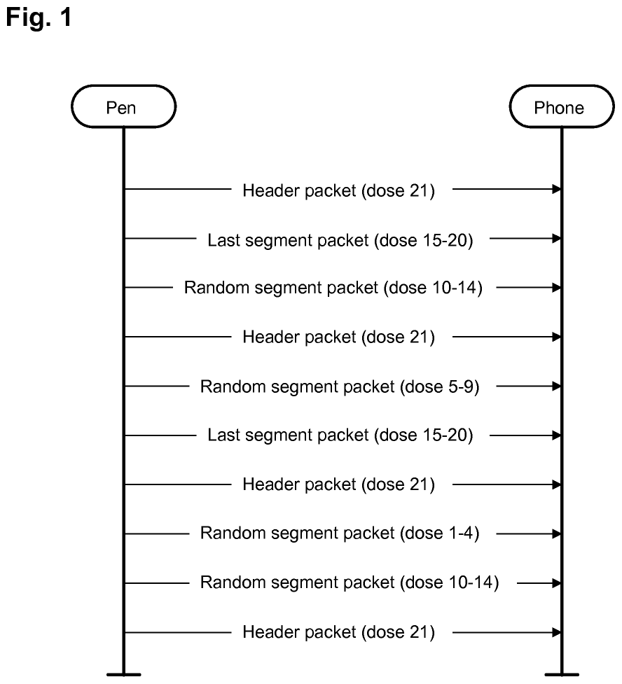

FIG. 1 illustrates an example where transmission of a dose log with 21 records is performed.

The advertising interval is a compromise between battery life and responsiveness. When a pen device is idle a slow interval is used and as soon as a dose delivery is performed the interval is significantly reduced for a period of time, e.g. 5 minutes.

Table 1 illustrates the expected average transfer time under perfect, typical, and worst-case radio conditions when using an advertising interval of 0.25 s for the active mode.

TABLE 1
Response times after dosing (packet interval T = 0.25 s)

| | Ideal | | Typical | | | |
|---|---|---|---|---|---|---|
| | conditions | | conditions | | Range limit | |
| | Maximum time | | Average time | | Average time | |
| Bit error rate (BER) | 0 | ppm | 100 | ppm | 1000 | ppm |
| Time to last dose | 0.75 | s | 0.4 | s | 0.7 | s |
| Time to last 6 doses | 1.25 | s | 0.7 | s | 1.2 | s |

| | | | | | |
|---|---|---|---|---|---|
| Time to transfer a | 33 | s | 68 | s | 2 min 30 s |
| full log | | | | | |

The expected use case is that a user transfers data after at least every 6^{th} injection, in this case a typical response time of 0.7 s should be expected.

Choosing an idle advertising interval of 4 s (16 times) and taking into account that when the phone is idle the scan interval may be reduced to 10% of the time, the following response times can be expected:

TABLE 2
Response times, idle (packet interval T = 4 s, scan 10%)

| | Typical conditions | | Range limit | |
|---|---|---|---|---|
| | Average time | | Average time | |
| Bit error rate (BER) | 100 | ppm | 1000 | ppm |
| Time to last dose | 1 min 17 s | | 1 min 18 s | |
| Time to last 6 doses | 2 min 9 s | | 2 min 10 s | |
| Time to transfer a full log | 7 | hours | 10 | hours |

So, in background mode in most of the cases the relevant information is transferred in less than 3 minutes, as long as the device is within range. A complete log takes roughly 7 hours to transfer. If the phone is activated the transfer time is reduced to 43 minutes, if the device is kept activated instead the transfer time is 38 minutes but then you might as well activate the phone also and get it over with in under a minute as seen in Table 1.

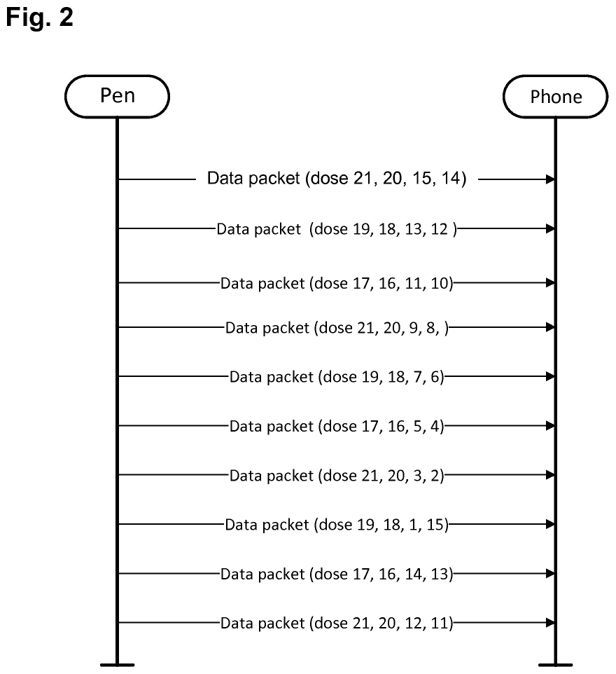

FIG. 2 illustrates an example of a transmission scheme using a single type of data packet (corresponding to the above-described header packet) where each data packet has room for four data entries in two groups of two. To save space the two entries in each group are in consecutive order (removes a dose id field). The first group (the prioritized group) is populated from the last 6 entries and the other group is populated from the reminder of the entries. In the figure the log contains 21 doses as the first example. The entries are selected in order to illustrate the principle but could of course be randomized.

For this example no probabilistic response time calculations have been performed but the maximum time in ideal conditions and 0.25 s packet interval would be:
Time to last dose: 0.75 s
Time to last 6 doses: 0.75 s
Time for full log: 37 s

It is to be noted that the ‘full log’ here and in the above tables are rare worst-case scenarios where the user empties the pen in one unit doses producing 300 entries without contact with the phone and then at the last dose starts the phone.

In the following a further exemplary embodiment will be described in which there are three packet types, segment, last segment, and header packets. Reference is made to an implementation in which user-set drug doses are expelled from a drug delivery device. The header packet contains the latest doses and various administrative data. The segment packet contains a segment of the dose log, and the last segment contains, in addition to a log segment, an authentication code for the whole event log.

The header packet contains the last 5 events, detailed timing for the last 5 doses, time, drug info, e.g. type of insulin, and a message authentication code for the header. It further contains a BLE header which may be a standard BLE advertising packet field header containing information about e.g. manufacturer, length of packet, and company. In a specific packet type field, the protocol and packet type within the company (e.g. Novo Nordisk A/S) is identified. Each log event is assigned a unique identifier. The first event record is assigned 1 and each new record get identifier numbers in consecutive order. A Last event ID field contains the id of the last record. It can also be seen as number of event records. A header packet message authentication code is calculated using OMAC.

The segment packet contains a ‘segment’ of the event log, e.g. up to 12 event records. In addition, it contains a BLE header (see above) as well as a type field and an ID field containing the event ID number for the last event in the segment packet.

The last segment packet contains the last segment of the event log (e.g. 6 event records) preceding the event records in the header record and a message authentication code for the whole event log. In addition, it contains a BLE header as well as a type field and an ID field containing the event ID number for the last event in the segment packet.

Each dose (event) record comprises a dose amount value, e.g. the size of an expelled dose measured in number of expelling mechanism increments (e.g. corresponding to “clicks”), and a timestamp. A dose extension record may be created to indicate non-regular events, e.g. undetermined dose size or detection of an air gap event.

In the following examples for packet scheduling will be described in which the following notation is used for packet types: H—Header packet, S—a segment packet, and L—a last segment packet. To indicate a particular packet, the ID/last ID field can be indicated as a subscript, e.g. H₃₀₀ indicates a header packet with last ID 300.

In the exemplary embodiment dose log transmission is implemented for a system, e.g. a drug delivery device, operating in four modes, each with a separate packet scheduling scheme: storage, idle, active and bulk.

In storage mode the device is in deep sleep and not transmitting at all. In idle mode between doses only header packets are transmitted, e.g. at an interval of 8 s. Active transmission mode occurs for 5 minutes after an injection. The packets are transmitted with an interval of 200 ms. The dose log is transferred with a header packet, a last packet and as many segment packets needed to transfer all records. For example, a full log (needing 29 segment packets) is transferred as follows:
HLSSSSSSSSSSSSSSSSSSSSSSSSSSSSS.

This is one round of transmission. The rounds are repeated until the active mode timeout (5 min) expires. In an exemplary embodiment at least every 10^{th} packet is a header packet. This gives a last dose response time of slightly more than 2 s at the cost of a 10% reduction in bulk transfer time for event logs larger than 115 records. In the above sequence a header packet is inserted after every 9^{th} packet and the round looks like this:
HLSSSSSSSSHSSSSSSSSSHSSSSSSSSSHSSS.

For shorter logs the number of segment packets is reduced to only as many segment packets necessary to transfer all records in the event log. The header packets may be distributed differently within a round as long as the maximum distance between two headers is always less than 10 and the number of packets is the same. For example, in the full log round the header packets could be distributed more evenly, like this:
HLSSSSSSSHSSSSSSSSHSSSSSSSHSSSSSSS.

To increase transmission efficiency also for the data entries in the last segment packet the last segment packet may be transmitted at a higher frequency, like this:
HSSSSLSSSSHSSSSSSSSHSSSSLSSSSHSSSSSSSS.

As appears, in each active mode the entire log is transmitted a number of times. However, in most cases it can be expected that the receiving device will have received and stored data during previous active modes, this allowing the log to be updated and completed by successfully receiving the header packet only. In contrast, some users may not be interested in the logging data for their personal use, but will merely transfer the entire log from a fully used pen device prior to a visit to a health care person.

The following table illustrates some concrete round sequences for various dose sizes (shown without randomized order). T_{H}/T is the average waiting time in package intervals (ideal radio conditions). T_{R}/T is the corresponding waiting time for a complete round.

TABLE 3
Packet sequences at various event log sizes

| Last | | | | |
|---|---|---|---|---|
| ID | Round sequence | T_{H}/T | T_{R}/T | Comment |
| 0 | H₀₀₀ | 0.5 | 0.5 | New pen, (not transmitted) |
| 1 | H₀₀₁ | 0.5 | 0.5 | One to 5 records - only |
| | | | | headers |
| 5 | H₀₀₅ | 0.5 | 0.5 | |
| 6 | H₀₀₆ L₀₀₁ | 1.0 | 1.5 | 6-11 records - a header |
| | | | | and a last segment packet |
| 7 | H₀₀₇ L₀₀₂ | 1.0 | 1.5 | |
| 11 | H₀₁₁ L₀₀₆ | 1.0 | 1.5 | |
| 12 | H₀₁₂ L₀₀₇ S₀₀₁ | 1.5 | 2.5 | 12-23 records - header, |
| | | | | last and 1 segment |
| 13 | H₀₁₃ L₀₀₈ S₀₀₂ | 1.5 | 2.5 | |
| 23 | H₀₂₃ L₀₁₈ S₀₁₂ | 1.5 | 2.5 | |
| 24 | H₀₂₄ L₀₁₉ S₀₁₃ S₀₀₁ | 2.0 | 3.5 | 24-35 records - header. |
| | | | | last and 2 segments |
| 25 | H₀₂₅ L₀₂₀ S₀₁₄ S₀₀₂ | 2.0 | 3.5 | |
| 60 | H₀₆₀ L₀₅₅ S₀₄₉ S₀₃₇ | 3.5 | 6.5 | A typical full log needs 7 |
| | S₀₂₅ S₀₁₃ S₀₀₁ | | | packets for a round (1.4 s) |
| 150 | H₁₅₀ L₁₄₅ S₁₃₉ S₁₂₇ | 4.2 | 14.5 | A large full log needs 15 |
| | S₁₁₅ S₁₀₃ S₀₉₁ S₀₇₉ | | | packets for a round (3.0 s). |
| | S₀₆₇ S₀₅₅ H₁₅₀ S₀₄₃ | | | Note the extra header record. |
| | S₀₃₁ S₀₁₉ S₀₀₇ | | | |
| 349 | H₃₄₉ L₃₄₄ S₃₃₇ S₃₂₅ | 4.6 | 33.5 | Almost full log |
| | S₃₁₃ S₃₀₁ S₂₈₉ S₂₇₇ | | | |
| | S₂₆₅ S₂₅₃ H₃₄₉ S₃₄₁ | | | |
| | S₂₂₉ S₂₁₇ S₂₀₅ S₁₉₃ | | | |
| | S₁₈₁ S₁₆₉ S₁₅₇ S₁₄₅ | | | |
| | H₃₄₉ S₁₃₃ S₁₂₁ S₁₀₉ | | | |
| | S₀₉₇ S₀₈₅ S₀₇₃ S₀₆₁ | | | |
| | S₀₄₉ S₀₃₇ H₃₄₉ S₀₂₅ | | | |
| | S₀₁₃ S₀₀₁ | | | |
| 350 | H₃₅₀ L₃₄₄ S₃₃₈ S₃₂₆ | 4.6 | 33.5 | Full log - 34 packets/round |
| | S₃₁₄ S₃₀₂ S₂₉₀ S₂₇₈ | | | (6.8 s) |
| | S₂₆₈ S₂₅₄ H₃₅₀ S₂₄₂ | | | |
| | S₂₃₀ S₂₁₈ S₂₀₆ S₁₉₄ | | | |
| | S₁₈₂ S₁₇₀ S₁₅₈ S₁₄₆ | | | |
| | H₃₅₀ S₁₃₄ S₁₂₂ S₁₁₀ | | | |
| | S₀₉₈ S₀₈₆ S₀₇₄ S₀₆₂ | | | |
| | S₀₅₀ S₀₃₈ H₃₅₀ S₀₂₆ | | | |
| | S₀₁₄ S₀₀₂ | | | |
| 400 | H₄₀₀ L₃₉₄ S₃₈₈ S₃₇₆ | 4.6 | 33.5 | Overflowed log - only 350 |
| | S₃₈₄ S₃₅₂ S₃₄₀ S₃₂₈ | | | records retained so 50 |
| | S₃₁₈ S₃₀₄ H₄₀₀ S₂₉₂ | | | records are lost unless |
| | S₂₈₀ S₂₆₈ S₂₅₆ S₂₄₄ | | | received earlier. Note |
| | S₂₃₂ S₂₂₀ S₂₀₈ S₁₉₆ | | | that only 2 event records |
| | H₄₀₀ S₁₈₄ S₁₇₂ S₁₆₀ | | | are valid on the oldest |
| | S₁₄₈ S₁₃₆ S₁₂₄ S₁₁₂ | | | segment packet (S₀₅₂) |
| | S₁₀₀ S₀₈₈ H₄₀₀ S₀₇₆ | | | |
| | S₀₆₄ S₀₅₂ | | | |

The segment packet (including last segment packet) ordering may be randomized for each new round as a way to somewhat mitigate periodic disturbances. When a new event is added to the log a new round is immediately started.

The following is an example of a possible packet sequence for 3 rounds of the 60 event case:
H₀₆₀ L₀₅₅ S₀₃₇ S₀₄₉ S₀₂₅ S₀₀₁ S₀₁₃ H₀₆₀ S₀₃₇ S₀₂₅ L₀₅₅ S₀₁₃ S₀₄₉ S₀₀₁ H₀₆₀ S₀₀₁ L₀₅₅ S₀₄₉ S₀₃₇ S₀₁₃ S₀₂₅

When a given drug delivery device has fully expelled the amount of drug for which it is designed, e.g. corresponding to 300 clicks being used in a drug delivery pen device, bulk transfer mode is used. The packet scheduling is the same as in active mode but the packet interval is the same as in idle mode (8 s).

The following table illustrates the theoretical response times that are expected for an experimental set-up under various conditions:

TABLE 4
Protocol candidate response/transfer times

| | Protocol mode | | | | | | | |
|---|---|---|---|---|---|---|---|---|
| | Active | | | | | | | |
| | (5 min after injection) | | | | Idle | | | |
| | Radio conditions | | | | | | | |
| | Typical | | Range | | Typical | | Range | |
| Advertising | 0.2 s | | | | 8 s | | | |
| interval | | | | | | | | |
| Receiver | 90% | | 90% | | 90% | | 10% | |
| duty cycle | | | | | | | | |
| Bit error | 100 | | 1000 | | 100 | | 1000 | |
| rate (ppm) | | | | | | | | |
| Measure | Avg | 98% | Avg | 98% | Avg | 98% | Avg | 98% |
| Last doses | 1.2 | 3.8 | 2.1 | 7.6 | 5.3 | 16 | 115 | 450 |
| (max log) | | | | | | | 2 min | 7 min |
| 5 events | | | | | | | | |
| ~2-4 doses | | | | | | | | |
| Typical | 2.1 | 4.2 | 4.1 | 9 | n/a | n/a | n/a | n/a |
| full log | | | | | | | | |
| 60 events | | | | | | | | |
| ~50 doses | | | | | | | | |
| Large | 5.3 | 10 | 11 | 21 | n/a | n/a | n/a | n/a |
| full log | | | | | | | | |
| 150 events | | | | | | | | |
| ~100 doses | | | | | | | | |
| Absolute | 15 | 27 | 30 | 55 | n/a | n/a | n/a | n/a |
| maximum | | | | | | | | |
| 350 events | | | | | | | | |

After having described an exemplary embodiment of the invention, a number of drug delivery devices incorporating the above-described transmission protocol will be described.

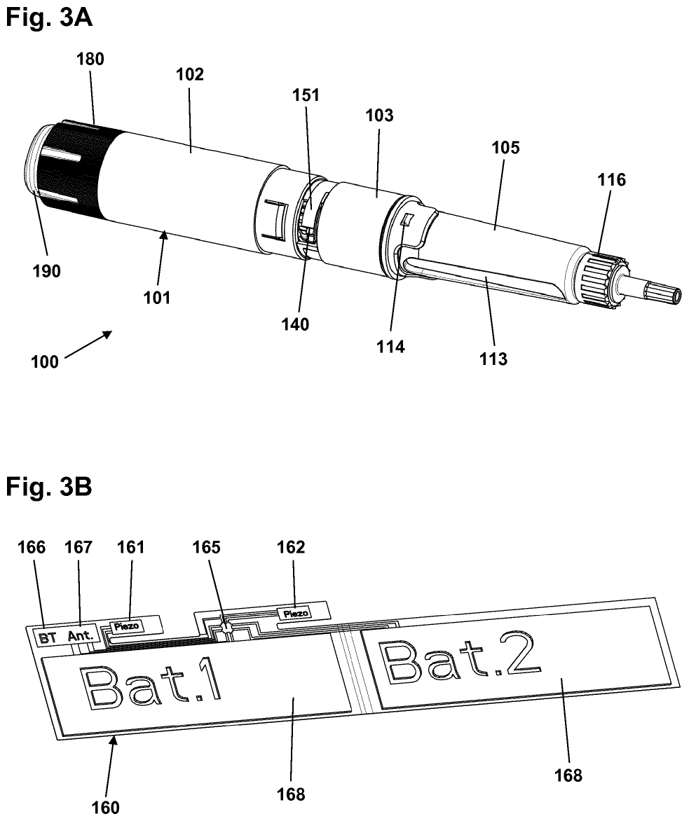

The pen device 100 in FIG. 3A comprises a proximal body or drive assembly portion with a housing 101 in which a drug expelling mechanism is arranged or integrated, and a distal cartridge holder portion in which a drug-filled transparent cartridge 113 with a distal needle-penetrable septum is arranged and retained in place by a non-removable cartridge holder attached to the proximal portion. The cartridge holder comprises openings allowing a portion of the cartridge to be inspected, distal coupling means allowing a needle assembly 116 to be releasably mounted as well as proximal coupling means in the form of two opposed protrusions 114 allowing a cap (not shown) to be releasably mounted covering the cartridge holder. In the shown embodiment the housing comprises a proximal housing portion 102 and a distal housing portion 103 which in a fully assembled state of the pen device is fixedly connected to each other via an intermediate tubular housing portion (not shown) covering the shown flexible arm 150 (see below), thereby forming a unitary housing. The cartridge is provided with a piston driven by a piston rod forming part of the expelling mechanism and may for example contain an insulin, GLP-1 or growth hormone formulation. A proximal-most rotatable dose setting member 180 serves to manually set a desired dose of drug and which can then be expelled when the button 190 is actuated. The expelling mechanism comprises a helically rotatable scale drum member with a plurality of indicia in the form of dose size numerals printed thereon, the dose size number corresponding to the currently set dose size being shown in a display opening (not seen in FIG. 3A). Depending on the type of expelling mechanism embodied in the drug delivery device, the expelling mechanism may comprise a spring as in the shown embodiment which is strained during dose setting and then released to drive the piston rod when the release button is actuated. Alternatively, the expelling mechanism may be fully manual in which case the dose member and the actuation button may be arranged to move proximally during dose setting corresponding to the set dose size, and then to be moved distally by the user to expel the set dose, e.g. as in a FlexPen® manufactured and sold by Novo Nordisk A/S.

Although FIG. 3A shows a drug delivery device of the prefilled type, i.e. it is supplied with a premounted cartridge and is to be discarded when the cartridge has been emptied, in alternative embodiments the drug delivery device may be designed to allow a loaded cartridge to be replaced, e.g. in the form of a “rear-loaded” drug delivery device in which the cartridge holder is adapted to be removed from the device main portion, or alternatively in the form of a “frontloaded” device in which a cartridge is inserted through a distal opening in the cartridge holder which is non-removable attached to the main part of the device.

The expelling mechanism incorporated in pen device 100 comprises a ring-formed piston rod drive element and an actuator member 140 in the form of a rotatable component that rotates together with the piston rod drive element during expelling of a dose of drug, the actuator member 140 thereby experiencing unidirectional rotational movement relative to an indicator structure fixedly disposed within the housing 101. In the shown embodiment the indicator structure is in the form of a pair of opposed circumferentially arranged deflectable flexible arms 151 each engaging the actuator member.

The actuator member 140 is in the form of a toothed wheel having a plurality of axially oriented ridges protruding radially outwards and being spaced circumferentially and equidistantly. Each ridge is formed with a gradually rising leading side and a sharply dropping trailing side. In the shown embodiment 24 ridges are spaced with angular steps of 15 degrees. Between any two neighbouring ridges a groove is formed.

Each of the deflectable arms 151 includes at its free end a tip portion with a radially inwards pointing first surface which is angled to be generally parallel with a gradually rising side of a ridge. Each tip portion further has a second opposed surface which is angled to be generally parallel with the sharply dropping side of a ridge. The radially inwards pointing first surface of the tip portions is configured to ride over consecutive ridges as the actuator member 140 rotates relative to the deflectable arms so that the tip portions of the first and second deflectable arm remain in intimate contact with the outer contour of the actuator member 140 as the latter rotates. The free end of a flexible arm 151 is biased slightly inwards when the tip portion is seated in a groove, the biasing force increasing when the free end of the arm is lifted outwards by the ridge formations as the actuator member rotates.

In the shown embodiment, the tip portions of the deflectable arms are located approximately 178 degrees apart so that, as the actuator member 140 rotates, the first deflectable arm will experience cooperation with a particular first ridge slightly before the second deflectable arm will experience cooperation with a ridge arranged diametrically opposite from the first protrusion. This arrangement is described in greater detail in EP application 17205309 hereby incorporated by reference. Alternatively, a single arm design may be used.

To monitor operation of the device by electronic means, electronic circuitry 160 is disposed in or on the device 100 for registering events associated with operations performed by the device, i.e. expelling of a set dose of drug. In the shown embodiment of FIG. 3B the electronic circuitry 160 is in the form of a flexible sheet on which is formed and mounted input means adapted to be actuated, directly or indirectly, by movement of the indicator structure(s), a processor and memory 165, wireless communication means 166 with antenna 167, and an energy source 168, wherein the processor is adapted to determine on the basis of measured values from the input means a rotational position and/or a rotational movement of the actuator member 140 to thereby calculate the size of an expelled dose of drug. The flexible sheet is adapted to be mounted on housing parts of the pen device by e.g. adhesive means, the nature of the flexible sheet allowing it to be mounted also on curved surfaces.

In the shown embodiment the input means is active transducers in the form of piezoelectric sensors 161, 162 adapted to be mounted onto the flexible arms 151 and thereby generating an output as the flexible arms are moved by the rotating actuator member 140. Although not incorporated in the shown embodiment, the electronic circuitry may in other embodiments further include a display so as to offer a visible read-out of information related to registered events. In the shown embodiment energy is provided by two electric cells 168.

One or more of the above-described components may be printed onto the flexible sheet, e.g. the piezoelectric sensors, a display, the antenna and the energy source. Other components, e.g. the processor and associated memory as well as a BLE radio chip may be surface mounted on the flexible sheet.

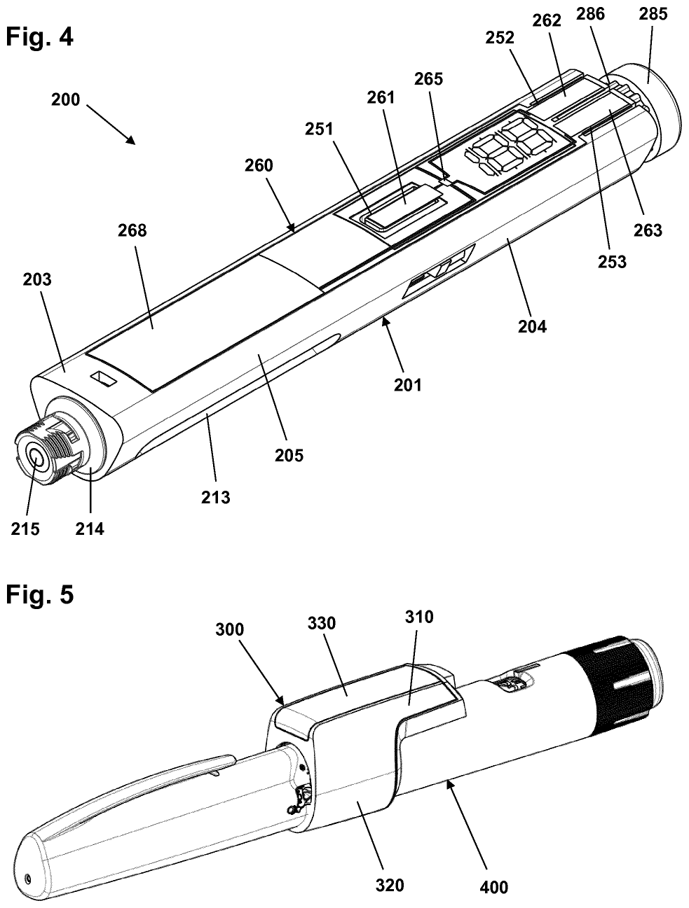

Turning to FIG. 4 a further pen device 200 incorporating electronic circuitry for the generation of a dynamic dose log will be described. The pen device 100 of FIG. 3A could be considered a traditional drug delivery device provided with electronic circuitry for the creation and transmission of a dose log, the pen device having a traditional user interface and being operated by a user in a traditional way, i.e. setting a dose size while observing a mechanical scale drum. In contrast, the pen device 200 is provided with a digital display replacing the traditional scale drum.

More specifically, the pen device 200 comprises a cylindrical housing 201 having a slightly curved information display surface 203 and a more conventionally curved opposing surface 204. The device is shown without a covering foil label, this allowing the electronic circuitry to be seen. The housing accommodates a drug containing cartridge 213, which has been inserted through an opening at a distal end thereof. The cartridge, which is closed at its distal end by a penetrable self-sealing septum 215 and at its proximal end by a slidable piston (not visible), is arranged in the distal cartridge holder portion 205 of the housing, being snapped to a proximal interior surface of the housing 201 by a snap coupling formed as part of the cartridge needle mount member 214 serving as an attachment interface for an injection needle unit (not shown). The housing is provided with a longitudinal window 206 for inspection of the cartridge contents and further accommodates both a dose setting mechanism and a drug expelling mechanism. The dose setting and expelling mechanism may be of any suitable design, e.g. a spring-driven design as shown, albeit without a scale drum. In the shown embodiment dose setting and dose release is performed using a combined dose setting and dose release member 285, i.e. the combined member is adapted to both rotate relative to the housing 201 during dose setting and to be moved axially to release a set dose.

As in the above-described embodiment, the expelling mechanism comprises an actuator member in the form of a rotatable component that rotates together with the piston rod drive element during expelling of a dose of drug, the actuator member thereby experiencing unidirectional rotational movement relative to an indicator structure fixedly disposed within the housing 201. In the shown embodiment the indicator structure is in the form of an axially arranged deflectable flexible arm 150 engaging the actuator member.

The combined dose setting and release member 285 extends into the housing 201 from a proximal end thereof. The combined member 285 comprises a cylindrical main body which is rotatable about a longitudinal axis of the housing. An axially grooved smaller-diameter actuator collar 286 is provided just distally of the main body and extends into the housing. The grooves have a spacing of 15 degrees and serve as actuators for dose setting input means, each groove corresponding to an increment of one dose unit, i.e. typically 1 IU of insulin.

In the housing 201 central portion some wall material has been removed to provide the abovementioned radially deflectable flexible dose expelling arm 250, and in a proximal portion wall material has been removed to provide first and second radially deflectable dose setting arms 251, 252, the latter being actuated by the grooved actuator collar 286. As described in greater detail in application EP2017/077850 the two dose setting arms allow incremental up/down rotation of the combined member 285 to be determined, this in turn being used to control the display to show the presently set dose size.

To monitor operation of the device by electronic means, electronic circuitry 260 is disposed on the device 200 for registering events associated with operations performed by the device, i.e. expelling of a set dose of drug. In the shown embodiment the electronic circuitry 260 is in the form of a flexible sheet on which is formed and mounted input means adapted to be actuated by movement of the indicator structures 250, 251, 252, a processor with memory and wireless communication means 265, a display 269 and an energy source 268, wherein the processor is adapted to determine on the basis of measured values from the input means a rotational position and/or a rotational movement of the actuator member to thereby calculate the size of an expelled dose of drug. The flexible sheet is adapted to be mounted on the curved housing surface 203 of the pen device by e.g. adhesive means.

In the shown embodiment the input means is active transducers in the form of piezoelectric sensors 261, 262, 263 adapted to be mounted onto the flexible arms 251, 252, 253 and thereby generating an output as the flexible arms are moved by the rotating actuator member respectively the dose setting actuator collar 286.

One or more of the above-described components may be printed onto the flexible sheet, e.g. the piezoelectric sensors, the display, an antenna and the energy source in the form of an electric cell. Other components, e.g. the processor and associated memory as well as a BLE radio chip may be surface mounted on the flexible sheet.

A further type of a drug delivery device comprising integrated dose logging circuitry is in the form of a traditional manual (i.e. non-spring-driven) drug delivery device in which the dose setting and actuation button will extend axially from the device as a dose is being set, the dose logging circuitry being arranged in the dose setting button and comprising e.g. a traditional rotary encoder adapted to register rotation during dose setting and/or dose expelling. A specific example of such a device is sold and manufactured by Novo Nordisk A/S as the NovoPen® 6, a pen device provided with wireless communication means allowing dose log data to be transferred to an external device using the above-described transmission protocol. NovoPen® 6 is provided with a display, however, this feature could alternatively be dispensed with.

A further example of how a drug delivery device can be provided with dose logging circuitry is disclosed in WO 2014/128155, hereby incorporated by reference, relating to an electronic logging unit adapted to be housed in a drug-filled cartridge having an axially displaceable piston and an outer cavity formed between the piston and the cartridge proximal opening, the logging unit comprising a general axis, a first distal portion adapted to engage the cartridge piston, and a second proximal portion adapted to engage a rotating element having a rotational axis corresponding to the general axis. The unit is provided with sensor means adapted to detect the amount of relative rotation between the first and second portions, storage means adapted to store data representing detected amounts of relative rotation, as well as transmitter means allowing the data to be transmitted to an external device. By this arrangement a logging unit can be provided in an essentially un-modified drug delivery device comprising a piston rod rotating during dose delivery, the rotation being transferred to the proximal portion of the logging unit, the latter being rotationally locked to the cartridge piston. This said, it may be necessary to use a cartridge with a more distally arranged piston to make room for the logging unit. The logging unit may be provided as an “ad-on” allowing a conventional durable, i.e. re-usable, drug delivery device to be provided with a logging functionality when needed. For example, when initiating a given patient on an insulin regimen the prescribing doctor may provide the patient with a drug delivery device in which a logging unit has been inserted, this allowing the doctor to check to which degree the patient has been in compliance with the regiment when the device is returned to the doctor after use. Indeed, the same logging unit could be used on a regular basis by any patient for which the logging capability and user interface are desirable. Alternatively, the logging unit could be provided in a disposable, pre-filled device.

Turning to FIG. 5 an add-on dose logging device 300 mounted on a drug delivery pen device 400 of the spring-driven type is shown, the add-on device incorporating electronic circuitry for the generation of a dynamic dose log when mounted on the pen device. In the present context the device represents a “generic” drug delivery device providing a specific example of a device in combination with which embodiments of the present invention can be used.

The logging module 300 comprises a body portion 310 and a ring-formed portion 320 allowing the add-on device to be mounted on a generally cylindrical pen device. The body portion comprises electronic circuitry and sensor means allowing a property to be detected representing an amount of drug being expelled from the cartridge, as well as an optional display 330 for displaying data to a user. The ring portion comprises coupling means allowing the add-on device to be securely and correctly mounted on the pen body. The electronic circuitry and the sensor means may in part be arranged in the ring portion.

The pen device comprises an indicator element with a magnet rotating together therewith during expelling of a dose of drug, the magnet being configured to generate a spatial magnetic field which relative to the sensor means varies corresponding to the spatial position and orientation of the magnet. The add-on device comprises sensor means adapted to measure a magnetic field as well as processor means configured to determine based on measured values rotational movement and/or positions of the indicator element based on which a dose log can be created. An exemplary embodiment of both the add-on device and the pen device is described in greater detail WO 2014/161952 which is hereby incorporated by reference. Additionally, the shown add-on device 300 is provided with wireless communication means allowing dose log data to be transferred to an external device using the above-described transmission protocol.

A further example of an add-on dose logging device adapted to be mounted on a drug delivery pen device of the spring-driven type is shown is shown in PCT/EP2018/075639, hereby incorporated by reference.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

In the above disclosure aspects of the present invention has described based on implementation in a drug delivery device of the pen type typically used to inject drugs having a blood glucose controlling effect, e.g. human insulin and analogues thereof as well as non-insulins such as GLP-1 and analogues thereof, as well as other types of drug, e.g. growth hormone or drugs for haemophilia treatment. Alternatively, the drug delivery device may be in the form of a body-worn drug infusion pump for e.g. insulin formulations.

However, these are only exemplary implementations. For example, aspects of the present invention may be implemented in a sensor device adapted to be mounted e.g. on a skin surface and adapted to measure and log a physiological parameter such as blood glucose values or skin temperatures. Alternatively, the sensor device may be in the form of a device adapted to be implanted, e.g. a pacemaker adapted to measure and log electrocardiographic values.

1. A method for wirelessly communicating a dynamic data log from a data generating device using a transmit-only protocol, the dynamic data log comprising:
a most-recent data entry,
a plurality of previous data entries,
the method comprising:
continuously or intermittently transmitting the dynamic data log as a plurality of data packets,
wherein the data packets comprise:
a prioritised packet populated by the most-recent data entry, and
a plurality of regular segment packets, each being populated with a subset of the plurality of previous data entries,
wherein the prioritised packet is transmitted more frequently than at least one of the regular segment packets.

2. The method as in claim 1, wherein the dynamic data log further comprises:
at least one recent data entry generated immediately before the most-recent data entry,
wherein the prioritised packet is populated by the most-recent data entry and the at least one recent data entry.

3. The method as in claim 1, wherein:
the prioritised packet and the regular segment packets are transmitted according to a predetermined order.

4. The method as in claim 1, wherein:
the regular segment packets are transmitted according to a dynamic order, the dynamic order being randomized or determined based on the number of data entries in the dynamic data log.

5. The method as in claim 1, wherein:
the regular segment packets are populated with a subset of the plurality of previous data entries according to a predetermined order.

6. The method as in claim 1, wherein:
for each transmission the population of the regular segment packets with previous data entries is randomized.

7. The method as in claim 2, wherein:
the dynamic data log further comprises at least one last data entry generated immediately before the at least one recent data entry,
the data packets further comprise one or more last segment packet populated by the at least one last data entry,
the plurality of regular segment packets each is populated with a subset of the data entries not included in the prioritised packet or the last segment packet, and
each last segment packet is transmitted more frequently than at least one of the regular segment packets.

8. The method as in claim 1, wherein the prioritised packet comprises a message authentication code for at least one of:
the prioritized packet, and
the whole data log.

9. The method as in claim 7, wherein the last segment packet comprises a message authentication code for at least one of:
the last segment packet, and
the whole data log.

10. The method as in claim 1, wherein a last segment packet and/or a regular segment packet are transmitted only when being populated with at least one data entry.

11. The method as in claim 1, wherein the prioritised packet is in the form of a header packet further comprising data indicating one or more of:
identity of the data generating device,
properties of the data generating device, and
properties and/or type of the data entries.

12. The method as in claim 1, wherein:
transmission of data packets takes place in an active mode and an idle mode,
in the active mode the entire data log is transmitted, the data packets being transmitted at a first rate, and
in the idle mode only the prioritised packet is transmitted, the prioritised packet being transmitted at a second rate, the second rate being lower than the first rate.

13. A drug delivery device comprising:
a drug reservoir or structure for receiving a drug reservoir,
drug expelling structure comprising dose setting structure allowing a user to set a dose amount of drug to be expelled, and
electronic circuitry adapted to create a dynamic data log related to expelled dose amounts of drug, comprising:
sensor structure adapted to capture a property value related to the dose amount of drug expelled from a reservoir by the expelling structure during an expelling event,
storage structure adapted to store a plurality of property values to create the dynamic log, the dynamic log comprising, with a sufficient amount of property values having been created:
at least one latest data entry, and
a plurality of previous data entries,
and
transmission structure for wireless transfer of the dynamic data log to an external device,
wherein the transmission structure is configured to transfer the dynamic data log using a transmit-only protocol as defined in claim 1.

14. A drug delivery device comprising:
a drug reservoir or structure for receiving a drug reservoir,
drug expelling structure comprising dose setting structure allowing a user to set a dose amount of drug to be expelled, and
electronic circuitry adapted to create a dynamic data log related to expelled dose amounts of drug, comprising:
sensor structure adapted to capture a property value related to the dose amount of drug expelled from a reservoir by the expelling structure during an expelling event,
storage structure adapted to store a plurality of property values to create the dynamic log, the dynamic log comprising, with a sufficient amount of property values having been created:
at least one latest data entry, and
a plurality of previous data entries,
and
transmission structure for wireless transfer of the dynamic data log to an external device,
wherein:
the transmission structure is configured to transfer the dynamic data log using a transmit-only protocol comprising:
transmission of data packets takes place in an active mode and an idle mode,
in the active mode the entire data log is transmitted, the data packets being transmitted at a first rate, and
in the idle mode only the prioritised packet is transmitted, the prioritised packet being transmitted at a second rate, the second rate being lower than the first rate,
the transmission structure can be operated in accordance with the active mode and the idle mode, and
the transmission structure is operated in the active mode for a predetermined amount of time after a data log entry has been created and stored, after which the transmission structure is operated in the idle mode.

15. A sensor device comprising:
sensor structure adapted to determine a physiological property value, and
electronic circuitry adapted to create a dynamic data log related to determined physiological property value, comprising:
storage structure adapted to store a plurality of physiological property values to create the dynamic log, the dynamic log comprising, with a sufficient amount of physiological property values having been created:
at least one latest data entry, and
a plurality of previous data entries,
and
transmission structure for wireless transfer of the dynamic data log to an external device,
wherein the transmission structure is configured to transfer the dynamic data log using a transmit-only protocol as defined in claim 1.
